(19) United States
(12) Patent Application Publication
(10) Pub. No.: US 2023/0065173 A1
(43) Pub. Date: Mar. 2, 2023

(54) CAUSAL RELATION INFERENCE DEVICE, CAUSAL RELATION INFERENCE METHOD, AND RECORDING MIDEUM

(71) Applicant: Hitachi, Ltd., Tokyo (JP)

(72) Inventors: Takuma SHIBAHARA, Tokyo (JP); Yasuho YAMASHITA, Tokyo (JP); Kunihiko KIDO, Tokyo (JP); Yoichi NAKAMOTO, Tokyo (JP); Shota NEMOTO, Tokyo (JP)

(21) Appl. No.: 17/863,542

(22) Filed: Jul. 13, 2022

(30) Foreign Application Priority Data
Sep. 2, 2021 (JP) 2021-142998

(51) Int. Cl. G06N 5/04; G06F 17/10

(52) U.S. Cl. CPC G06N 5/048; G06F 17/10

(57) ABSTRACT

To infer a value of an assignment variable with high accuracy. In a causal relation inference device including a processor configured to execute a program and a storage device storing the program, the processor is configured to execute a first calculation process of calculating an internal vector based on a feature vector of a plurality of samples and a first learning parameter, a second calculation process of calculating a reallocation vector based on a second learning parameter and the internal vector calculated by the first calculation process, and a third calculation process of calculating a pointwise weight vector for each of the plurality of samples based on a third learning parameter and the reallocation vector calculated by the second calculation process.

### CLAIM OF PRIORITY

The present application claims priority from Japanese patent application JP 2021-142998 filed on Sep. 2, 2021, the content of which is hereby incorporated by reference into this application.

### BACKGROUND OF THE INVENTION

### 1. Field of the Invention

The present invention relates to a causal relation inference device, a causal relation inference method, and a causal relation inference program for inferring a causal relation in data.

### 2. Description of the Related Art

In order to accurately estimate an effect of a therapy or a drug, it is necessary to compare a case of performing therapeutic or drug treatment on the same patient with a case of not performing the therapeutic or drug treatment. However, there are cases where it is impossible to perform experiments in which an operation with and an operation without administration are repeated to the same patient, such as anticancer drug treatment with strong side effects. Therefore, there is a method referred to as propensity score analysis as a method of estimating the treatment effect for convenience by comparing a patient group on which the treatment is performed and a patient group on which the treatment is not performed. With the propensity score analysis, the treatment effect can be estimated by comparing similar patients in the treatment group and the non-treatment group.

A propensity score is a value indicating a probability that a patient belongs to the treatment group. By estimating the effect of treatment or administration for patients with similar tendencies in belonging to the treatment group, it is possible to estimate an effect similar to that of a case of performing treatment on the same patient. Generally, a statistical method referred to as logistic regression is used as a method for calculating the propensity score, but it is difficult to identify similar patients since prediction accuracy of the logistic regression is low.

Deep Learning (neural network) is one of techniques for implementing artificial intelligence (AI). With Deep Learning, high prediction accuracy can be achieved. Each element of a feature vector, which is an input item used for prediction, is subjected to a weighted product-sum operation with other elements each time the element passes through a plurality of perceptrons. Therefore, it is difficult in principle to know importance of each element of the feature vector. This is a fatal drawback when Deep Learning is used in a medical field.

For example, if values of propensity scores of two patients are close to each other and the AI cannot explain a determination criteria, it is extremely difficult for a physician to determine whether the patients happen to have the same value or truly has similar properties.
Non-Patent Literature 1 (Friedman J, Trevor H, Robert T. The elements of statistical learning. second edition. New York: Springer series in statistics, 2001) discloses a method for newly learning linear regression or logistic regression such that an identification result of a machine learning method such as Deep Learning, which does not have a function of calculating importance of a feature, can be explained. The logistic regression is a machine learning model equivalent to the perceptron and is the most widely used in all fields. For example, the logistic regression shown on page 119 of Non-Patent Literature 1 (Friedman J, Trevor H, Robert T. The elements of statistical learning. second edition. New York: Springer series in statistics, 2001) has a function of calculating importance of a feature for the entire data sample.
Non-Patent Literature 2 (Ribeiro, Marco Tulio, Sameer Singh, and Carlos Guestrin. “Why should I trust you?: Explaining the predictions of any classifier.” Proceedings of the 22nd ACM SIGKDD International Conference on Knowledge Discovery and Data Mining. ACM, 1016) discloses a new explanation method for interpretably and faithfully explaining prediction of a classifier by learning an interpretable model that locally changes the prediction. This explanation method explains various models of text (such as a random forest) and image classification (such as a neural network).
Non-Patent Literature 3 (Golas, Sara Bersche, et al. “A machine learning model to predict the risk of 30-day readmissions in patients with heart failure: a retrospective analysis of electronic medical records data.” BMC medical informatics and decision making 18.1 (1018): 44) discloses a model to predict the risk of 30-day readmissions in patients with heart failure discharged from a hospital. This risk prediction model uses a deep unified network, which is a new mesh-like network structure for deep learning designed to avoid overfitting.
Non-Patent Literature 4 (Dehejia, Rajeev H., and Sadek Wahba. “Causal effects in nonexperimental studies: Reevaluating the evaluation of training programs.” Journal of the American statistical Association 94.448 (1999): 1053-1062) discloses a method for evaluating an effect of a vocational training program.

The method of Non-Patent Literature 2 is merely an attempt to make an explanation later by linear regression, and even in a case of trying to explain a normal fully connected type Deep Learning, there is no mathematical guarantee that importance of a feature used in prediction by Deep Learning can be completely calculated. If a perfect linear regression can achieve the same prediction accuracy as Deep Learning, the first Deep Learning itself is no longer necessary. Therefore, the method of Non-Patent Literature 1 has a contradiction in construction concept.

### SUMMARY OF THE INVENTION

In view of the problem described above, an object of the invention is to infer a value of an assignment variable with high accuracy.

A causal relation inference device according to one aspect of the invention disclosed in the present application, is a causal relation inference device including a processor configured to execute a program and a storage device storing the program. The processor executes a first calculation process of calculating an internal vector based on a feature vector of a plurality of samples and a first learning parameter, a second calculation process of calculating a reallocation vector based on a second learning parameter and the internal vector calculated by the first calculation process, and a third calculation process of calculating a pointwise weight vector for each of the plurality of samples based on a third learning parameter and the reallocation vector calculated by the second calculation process.

According to a representative embodiment of the invention, a value of an assignment variable can be inferred with high accuracy. Problems, configurations, and effects other than those described above are made clear by the following explanation of the embodiment.

### BRIEF DESCRIPTION OF THE DRAWINGS

FIG. 1 is a block diagram showing system configuration examples of a causal relation inference system.

FIG. 2 is a diagram illustrating a configuration example of a neural network.

FIG. 3 is a flowchart showing an example of a causal relation inference process procedure by a causal relation inference device.

FIG. 4 is a flowchart showing an example of a detailed process procedure of learning parameter generation (step S302).

FIG. 5 is a diagram illustrating an experimental result.

FIG. 6 is a diagram illustrating an importance distribution for each feature using kernel density estimation in the experimental result.

### DESCRIPTION OF THE PREFERRED EMBODIMENTS

### First Embodiment

In the first embodiment, a causal relation inference device that estimates an effect or efficacy of a drug and outputs what patient background and factors are used to identify a plurality of patients will be described as an example. By the first embodiment, a physician can accurately estimate an effect of a drug. This will contribute to swift recovery of a patient and improvement in quality of medical care, and will lead to reduction of medical expenses in a country, which is increasing at an accelerated pace, by reducing less-effective treatments.

### System Configuration Example

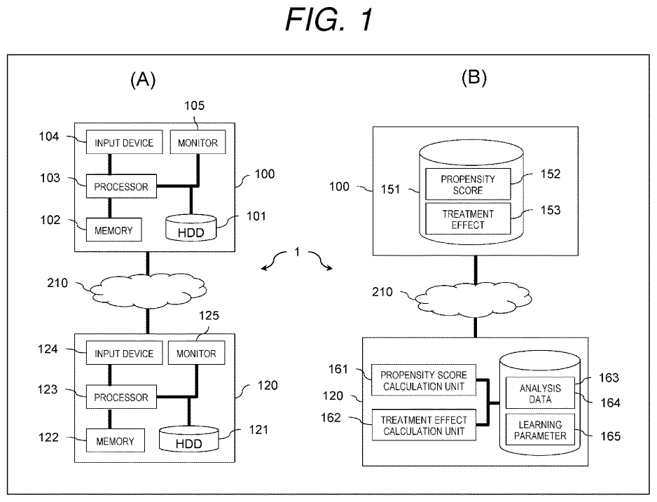

FIG. 1 is a block diagram showing system configuration examples of a causal relation inference system. In FIG. 1, a server-client type causal relation inference system 1 will be described as an example, but a stand-alone type causal relation inference system 1 may also be used. (a) of FIG. 1 is a block diagram showing a hardware configuration example of the causal relation inference system 1, and (b) of FIG. 1 is a block diagram showing a functional configuration example of the causal relation inference system 1. In FIG. 1, the same components are designated by the same reference numerals.

The causal relation inference system 1 is configured such that a client terminal 100 and a causal relation inference device 120, which is a server, are communicably connected via a network 110.

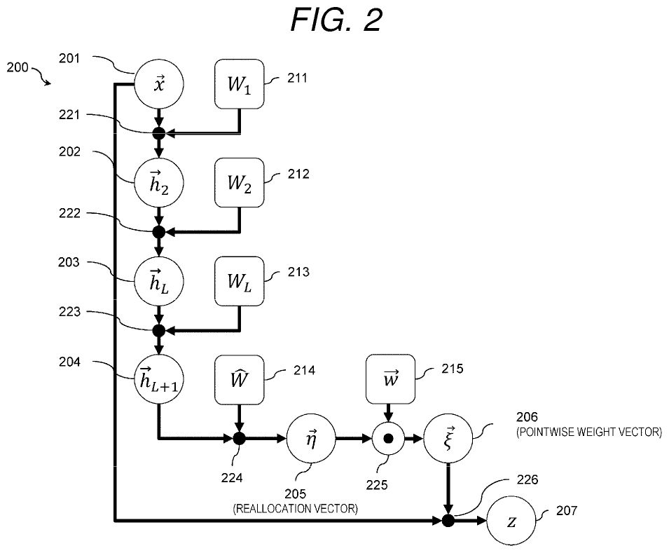

In (a) of FIG. 1, the client terminal 100 includes a hard disk drive (HDD) 101 which is an auxiliary storage device, a memory 102 which is a main storage device, a processor 103, an input device 104 which is a keyboard or a mouse, and a monitor 105. The causal relation inference device 120 includes an HDD 121 which is an auxiliary storage device, a memory 122 which is a main storage device, a processor 123, an input device 124 which is a keyboard or a mouse, and a monitor 125. It should be noted that the main storage device, the auxiliary storage device, and a portable storage medium (not shown) are collectively referred to as a storage device. The storage device stores a neural network 200 shown in FIG. 2 and these learning parameters 165.

In (b) of FIG. 1, the client terminal 100 includes a client database (DB) 151. The client DB 151 is stored in a storage device such as the HDD 101 or the memory 102. A propensity score 152 and a treatment effect 153 are stored in the client DB 151.

The propensity score 152 is an assignment variable, for example, a prediction value indicating the presence or absence of treatment, and is an example of a prediction value indicating a causal relation. The treatment effect 153 is information indicating a treatment effect. The treatment refers to, for example, an act of controlling the presence or absence or degree of a factor (including a behavior leading to maintenance and promotion of health, and administration and test for prevention, diagnosis or therapy of injuries and illnesses in medical treatment) that affect various events related to human health. The treatment effect 153 is data obtained from a treatment effect calculation unit 162 via the network 110. It should be noted that when the causal relation inference system 1 is a server-client type causal relation inference system 1, the number of the client terminals 100 is one or more.

The causal relation inference device 120 includes a propensity score calculation unit 161, the treatment effect calculation unit 162, and a server database (DB) 163. The propensity score calculation unit 161 calculates the propensity score 152 and generates the learning parameter 165.

The treatment effect calculation unit 162 constructs the neural network 200 by using the learning parameter 165, executes a prediction process by giving test data to the neural network 200, and outputs the treatment effect 153 to the client terminal 100. The propensity score calculation unit 161 and the treatment effect calculation unit 162 realize functions thereof by causing the processor 123 to execute a program stored in a storage device such as the HDD 121 and the memory 122. The server DB 163 stores analysis data 164 and the learning parameter 165.

It should be noted that a plurality of causal relation inference devices 120 may be used. For example, a plurality of causal relation inference devices 120 may be provided for the purpose of load distribution. In addition, a plurality of causal relation inference devices 120 may be used for each function. For example, the causal relation inference device 120 may include a first server including the propensity score calculation unit 161 and the server DB 163, and a second server including the treatment effect calculation unit 162 and the server DB 163. In addition, the causal relation inference device 120 may include a first causal relation inference device including the propensity score calculation unit 161 and the treatment effect calculation unit 162, and a second causal relation inference device including the server DB 163. Further, the causal relation inference device 120 may include a first causal relation inference device including the propensity score calculation unit 161, a second causal relation inference device including the treatment effect calculation unit 162, and a third causal relation inference device including the server DB 163.

### Configuration Example of Neural Network

FIG. 2 is a diagram illustrating a configuration example of a neural network. In the neural network 200, a feature vector 201 is a D-dimensional (D is an integer of 1 or more) real-valued vector, and includes a feature that is a factor value such as administration information and a test value for each age and gender. In the feature vector 201, as in Non-Patent Literature 3, a feature, which is a value of a 3,512-dimensional factor (D=3,512), is used.

In addition, the neural network 200 includes internal vectors 202 to 204. The neural network 200 outputs, to the internal vector 202, an operation result of a product-sum operation 221 of the feature vector 201 and a learning parameter 211.

In addition, the neural network 200 outputs, to the internal vector 203, an operation result of a product-sum operation 222 of an operation result of the internal vector 202 and a learning parameter 212.

In addition, the neural network 200 outputs, to the internal vector 204, an operation result of a product-sum operation 223 of an operation result of the internal vector 203 and a learning parameter 213.

In addition, the neural network 200 calculates a reallocation vector 205 which is a calculation result of a product-sum operation 224 of an operation result of the internal vector 204 and a learning parameter 214.

In addition, the neural network 200 calculates a pointwise weight vector 206 which is an operation result of a Hadamard operation 225 of the reallocation vector 205 and a learning parameter 215. The pointwise weight vector 206 is a weight vector having a different weight value for each patient as a sample.

In addition, the neural network 200 calculates, by a product-sum operation 226 of the feature vector 201 and the pointwise weight vector 206, a prediction value 207 for an assignment variable Z₍ₙ₎ indicating a predetermined action for each patient n, for example, the presence or absence of treatment, that is, the propensity score 152.

### Example of Causal Relation Inference Process Procedure

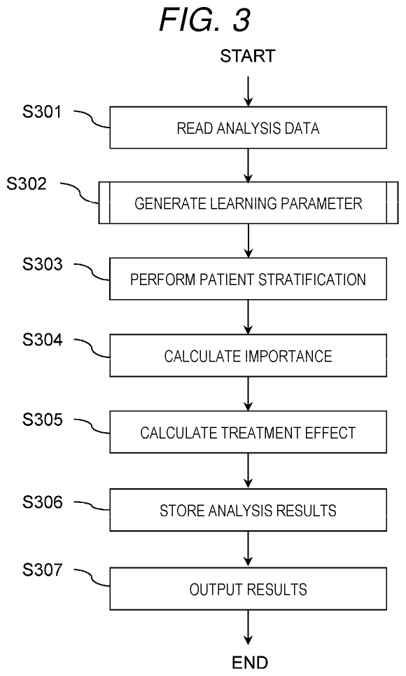

FIG. 3 is a flowchart showing an example of a causal relation inference process procedure by a causal relation inference device. In FIG. 3, steps S301 and S302 are processes by the propensity score calculation unit 161, and steps S303 to S307 are processes by the treatment effect calculation unit 162.

The causal relation inference device 120 reads the known analysis data 164 (step S301). The analysis data 164 is data including a combination of the feature vector 201 for each patient n, the assignment variable Z₍ₙ₎ for each patient n, and a treatment effect y₍ₙ₎ for each patient n. It should be noted that n={1, . . . , N} is an index for designating data of a certain patient, and in the first embodiment, N=30,000.

In order to improve the ease of understanding of the first embodiment, the feature vector 201 will be described by taking, as an example, three dimensions (D=3) of {age, gender, white blood cell count}. In addition, the assignment variable Z₍ₙ₎ is a target variable, that is, a correct label, and takes two values of “1” or Specifically, for example, Z₍ₙ₎=1 means “treatment”, and Z₍ₙ₎=0 means “non-treatment”.

Further, the treatment effect y₍ₙ₎ is a therapeutic effect after treatment is performed, such as a blood pressure after administration of an antihypertensive drug. It should be noted that the causal relation inference device 120 in the first embodiment calculates, as an example, an average therapeutic effect in patient populations of a group with treatment and a group without treatment.

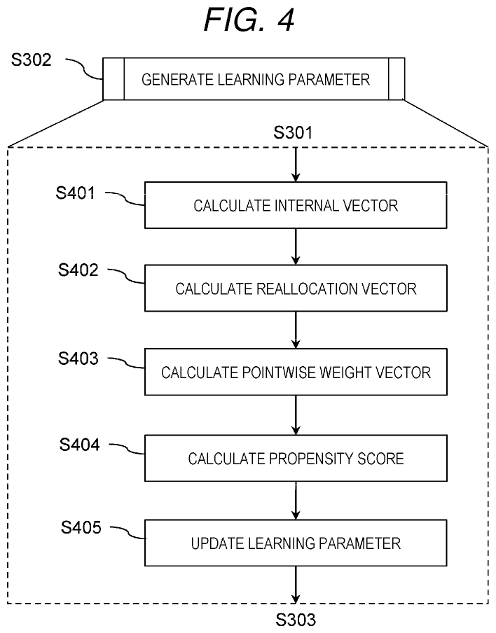

Next, the causal relation inference device 120 generates the learning parameter 165 (step S302). Here, details of the learning parameter generation (step S302) will be described with reference to FIG. 4.

FIG. 4 is a flowchart showing an example of a detailed process procedure of the learning parameter generation (step S302). First, the causal relation inference device 120 calculates an internal vector by the following Equation (1) (step S401).

\vec{h}ₗ₊₁=σ(Wₗ\vec{h}ₗ) Equation (1)
where Wₗ∈R^{\tilde{D}×\tilde{D}}

(in first embodiment, for example, \tilde{D}=100)

The vectors h₁, hₗ₊₁ in the above Equation (1) are the internal vectors 202, 203, and 204. l=1, . . . , and L is an index indicating the number of layers of an internal neuron group. In the first embodiment, L=2.

Wₗ on a right side of the above Equation (1) is the learning parameter 165 (211, 212, and 213). When l=1, the internal vector h₁ on the right side of the above Equation (1) is the feature vector 201, and Wₗ is the learning parameter 211.

σ on the right side of the above Equation (1) is a sigmoid function. Internal operations of the sigmoid function σ are the product-sum operations 221, 222, 223, 224, and 226 of the learning parameter 165 and the internal vectors, the product-sum operations 221, 222, 223, 224, and 226 forming a matrix.

It should be noted that a bias parameter and a patient index n are removed from the above Equation (1) in order to improve the ease of understanding the configuration of the first embodiment. Although the above Equation (1) shows a fully connected neural network, it is also possible to use an operation of a neural network such as a long short-term memory (LSTM) and a convolutional neural network (CNN).

Next, the causal relation inference device 120 calculates the reallocation vector 205 by the following Equation (2) (step S402).

\vec{η}=Ŵ\vec{h}_{L+1} Equation (2)

where Ŵ∈R^{D×\tilde{D}} represents learning parameter 214

η (∈R^{D}) on a left side of the above Equation (2) is the reallocation vector 205. Internal vector h_{L+1} on a right side of the above Equation (2) is a product-sum operation result of the internal vector 203 (h_{L}) and the learning parameter 213 (W_{L}).

Next, the causal relation inference device 120 calculates the pointwise weight vector 206 by the following Equation (3) (step S403).

\vec{ξ}=\vec{w}⊙\vec{η} Equation (3)

where ⊙ represents Hadamard product

A vector ξ (∈R^{D}) on a left side of the above Equation (3) is the pointwise weight vector 206. A vector w (∈R^{D}) on a right side of the above Equation (3) is the learning parameter 215. An operator on the right side of the above Equation (3) represents a Hadamard product, and executes the Hadamard operation 225.

Next, the causal relation inference device 120 calculates, by the following Equation (4), the propensity score 152, which is the prediction value 207 indicating the presence or absence of treatment (step S404).

z=σ(\vec{ξ}·\vec{x}) Equation (4)

z (0≤z≤1) on a left side of the above Equation (4) is the prediction value 207 for the assignment variable Z₍ₙ₎ indicating the presence or absence of treatment, that is, the propensity score 152. It should be noted that in the above Equation (4), when the treatment effects 153 of a plurality of classes are solved, a softmax function is used instead of the sigmoid function σ.

Next, the causal relation inference device 120 updates and optimizes the learning parameter 165 (211 to 215) by using a statistical gradient method such that a cross entropy shown in the following Equation (5) is minimized by using the assignment variable Z₍ₙ₎ (the presence or absence of treatment) and z₍ₙ₎ which is the propensity score 152 (the prediction value 207 for the assignment variable Z₍ₙ₎ indicating the presence or absence of treatment) (step S405). An initial value of the learning parameter 165 (211 to 215) is set by a random number.

argmin_{{W}_{ₗ}_{,Ŵ,\vec{w}}}Σₙ−(Z₍ₙ₎ log(z₍ₙ₎)+(1−Z₍ₙ₎)log(1−z₍ₙ₎)) Equation (5)

The causal relation inference device 120 stores, in the server DB 163, the generated learning parameter 165 (211 to 215). Accordingly, the learning parameter generation (step S302) is completed, and the process proceeds to step S303.

It should be noted that the learning parameter 165 updated by the learning parameter generation (step S302) is subsequently applied to the learning parameter generation (step S302) when the analysis data 164 is newly read (step S301).

Returning to FIG. 3, the causal relation inference device 120 performs patient stratification (step S303). Specifically, for example, the causal relation inference device 120 applies k-means clustering to the reallocation vector 205 (η) (where k>0). In the first embodiment, k=5 classes, and patient population C₍ₖ₎ of 5 classes is divided such that C₍ₖ₎={C₍₁₎, C₍₂₎, . . . , C₍₅₎} and this process is referred to as patient stratification. The patient index n is retained as in the patient population C₍ₖ₎={1, . . . , 33, . . . , 200, . . . }. In addition, in the first embodiment, distance L2 is used as a distance scale in a case of performing the k-means clustering, but the distance scale is not limited to distance L2.

As described above, by performing clustering using the reallocation vector 205, a patient population C₍ₖ₎ can be generated in patient groups having similar feature vectors 201 to which the learning parameter 165 is applied.

It should be noted that the causal relation inference device 120 may execute a patient stratification process by executing the k-means clustering using, instead of the reallocation vector 205, any of the pointwise weight vector 206, the feature vector 201, the propensity score 152, and a patient-specific importance vector p₍ₙ₎ calculated by the following Equation (6).

$${\vec{p}}_{( n )} = {\vec{ξ}}_{( n )} ⊙ {\vec{x}}_{( n )} - \frac{1}{N} {\sum }_{n} [ {\vec{ξ}}_{( n )} ⊙ {\vec{x}}_{( n )} ] \qquad Equation ( 6 )$$

As described above, by performing clustering by using the pointwise weight vector 206, a patient population C₍ₖ₎ can be generated in patient groups having similar pointwise weight vectors 206. Accordingly, compared with the case of using the reallocation vector 205, patients having the similar pointwise weight vectors 206 in the patient population C₍ₖ₎ generate a population having similar prediction models (Equation 4) that produce, for each patient, the z₍ₙ₎ which is the propensity score 152.

In addition, by performing clustering using the feature vector 201, a patient population C₍ₖ₎ can be generated in patient groups having similar feature vectors 201. Accordingly, compared with the case of using the reallocation vector 205, original properties of patients having similar feature vectors 201 in the patient population C₍ₖ₎ are aligned.

In addition, by performing clustering using the propensity score 152, a patient population C₍ₖ₎ can be generated in patient groups having similar propensity scores 152. Accordingly, compared with the case of using the reallocation vector 205, patients having similar prediction values 207 for the assignment variable Z₍ₙ₎ indicating the presence or absence of treatment in the patient population C₍ₖ₎ are conceptually aligned as patients having similar treatment probabilities.

In addition, by performing clustering using the importance vector p₍ₙ₎, a patient population C₍ₖ₎ can be generated in patient groups having similar importance vectors p₍ₙ₎. Accordingly, compared with the case of using the reallocation vector 205, patients having similar importance vectors p₍ₙ₎ in the patient population C₍ₖ₎ are aligned as patients having similar characteristics in calculating the propensity score 152.

The patient stratification (step S303) is a selectable process, and the process may skip the patient stratification (step S303) and proceed to importance calculation (step S304) after the learning parameter generation (step S302).

Next, the causal relation inference device 120 calculates an importance (step S304). Specifically, for example, the causal relation inference device 120 calculates a patient-specific importance vector p′₍ₘ₎(k) for each patient population C₍ₖ₎ by the following Equation (7). The importance vector p′₍ₘ₎(k) includes an importance of each factor of the feature vector 201 of a patient m in each patient population C₍ₖ₎.

$${{p}^{\to ′}}_{( m )} ( k ) = {\vec{ξ}}_{( m )} ⊙ {\vec{x}}_{( m )} - \frac{1}{{N}_{( k )}} {\sum }_{m} [ {\vec{ξ}}_{( m )} ⊙ {\vec{x}}_{( m )} ] \qquad Equation ( 7 )$$

N₍ₖ₎ on a right side of the above Equation (7) is the number of patients in the patient population C₍ₖ₎. For a patient with a certain index m, the patient-specific importance vector p′₍ₘ₎(k) is calculated in the patient population C₍ₖ₎ to which the patient belongs. It should be noted that an average importance vector of the patient population C₍ₖ₎ is set as p(k).

Next, the causal relation inference device 120 calculates the treatment effect 153 (step S305). Specifically, for example, the causal relation inference device 120 calculates, by the following Equation (8), an average treatment effect ATE (k) for each patient population C₍ₖ₎ as the treatment effect 153. If the treatment effect y₍ₙ₎ is a therapeutic result when treatment is performed, such as a blood pressure after administration of an antihypertensive drug, the average treatment effect ATE (k) is an average value of blood pressures after administration of the antihypertensive drug for each patient population C₍ₖ₎.

$$ATE ( k ) = \frac{{\sum }_{m ∈ {C}_{( k )}} {Z}_{( m )} {y}_{( m )} / {z}_{( m )}}{{\sum }_{m ∈ {C}_{( k )}} {Z}_{( m )} / {z}_{( m )}} - \frac{{\sum }_{m ∈ {C}_{( k )}} ( 1 - {Z}_{( m )} ) {y}_{( m )} / ( 1 - {z}_{( m )} )}{{\sum }_{m ∈ {C}_{( k )}} ( 1 - {Z}_{( m )} ) / ( 1 - {z}_{( m )} )} \qquad Equation ( 8 )$$

Then, the causal relation inference device 120 calculates, by the following Equation (9), an average value AATE of the average treatment effects ATE (k) for each patient population C₍ₖ₎ among all patient populations C₍₁₎, C₍₂₎, . . . , C₍ₖ₎.

$$AATE = \frac{1}{N} {\sum }_{k} {N}_{k} ATT ( k ) \qquad Equation ( 9 )$$

Nₖ on a right side of the above Equation (9) is the number of patients of class k.

Next, the causal relation inference device 120 stores, in the client DB 151, the propensity score 152 {ξ₍ₙ₎, p₍ₙ₎, p′₍ₘ₎(k), z₍ₙ₎, C₍ₖ₎} and the treatment effect 153 {ATE (k), AATE} as analysis results (step S306).

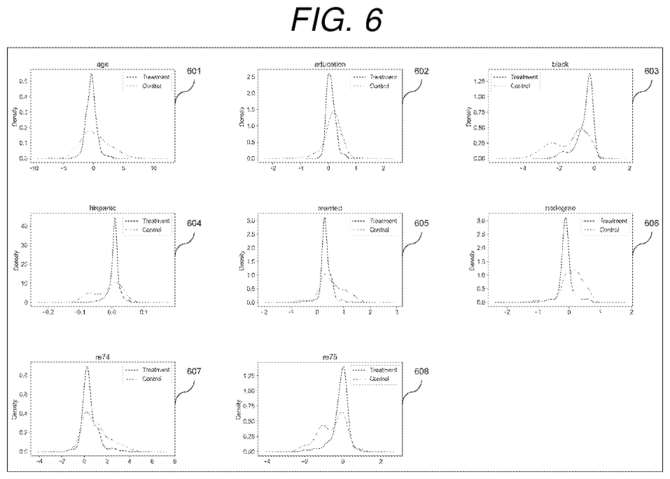

It should be noted that the causal relation inference device 120 may classify, in each of the patient populations C₍ₖ₎, the patient population C₍ₖ₎ into a treatment group and a non-treatment group for each factor of the feature vector 201 based on z₍ₙ₎ (the prediction value 207 for the assignment variable Z₍ₙ₎ indicating the presence or absence of treatment). Specifically, for example, the causal relation inference device 120 may set a patient n in the patient population C₍ₖ₎ whose z₍ₙ₎ (the prediction value 207 for the assignment variable Z₍ₙ₎ indicating the presence or absence of treatment) is equal to or greater than a threshold value as a treatment group for the factor, and set a patient n whose z₍ₙ₎ is smaller than the threshold as a non-treatment group for the factor. The causal relation inference device 120 may perform this classification on a patient population C₍ₖ₎ for which a particular treatment effect 153 is obtained. Then, the causal relation inference device 120 may compare the treatment group and the non-treatment group at the same importance for each factor, for example, by using FIG. 6 described later.

Next, the causal relation inference device 120 outputs the treatment effect 153 to the client terminal 100. The client terminal 100 displays the treatment effect 153 on the monitor 105.

As described above, according to the first embodiment, the causal relation inference device 120 calculates the pointwise weight vector 206 (ξ) for each patient n. Accordingly, the causal relation inference device 120 can infer, with high accuracy, z₍ₙ₎ (the prediction value 207 for the assignment variable Z₍ₙ₎ indicating the presence or absence of treatment) indicating whether treatment is performed on the patient n. Therefore, if the z₍ₙ₎ (the prediction value 207 indicating the presence or absence of treatment) of a patient in the treatment group and the z₍ₙ₎ (the prediction value 207 for the assignment variable Z₍ₙ₎ indicating the presence or absence of treatment) of a patient in the non-treatment group are equal (the values are the same or a difference between the values is within an allowable range), the patient in the non-treatment group is identified with the patient in the treatment group, and for example, it can be determined that the patient in the non-treatment group may receive the same treatment as the patient in the treatment group.

In particular, since a patient group is converged into a plurality of patient groups C₍ₖ₎ by patient stratification, treatment same as treatment to the patient in the treatment group can be performed on the patient in the non-treatment group as described above for each patient population C₍ₖ₎.

In addition, accuracy of causal relation inference can be improved by learning using the assignment variable Z₍ₙ₎ indicating the presence or absence of treatment and z₍ₙ₎ (the prediction value 207 for the assignment variable Z₍ₙ₎ indicating the presence or absence of treatment).

In addition, the causal relation inference device 120 can infer, by the pointwise weight vector 206 for each patient n, a patient-specific importance vector p′₍ₘ₎(k) indicating a causal relation between the feature vector 201 for each patient n which is a cause and the treatment effect y₍ₙ₎ for each patient n which is a result. Accordingly, it is possible to facilitate explanation of analysis assumption of the z₍ₙ₎ which is the propensity score 152 (the prediction value 207 for the assignment variable Z₍ₙ₎ indicating the presence or absence of treatment).

In addition, the causal relation inference device 120 can predict, with high accuracy, a result (the treatment effect 153 {ATE (k), AATE}) in the patient population by using the feature vector 201 which is the cause, the treatment effect y₍ₙ₎ for each patient which is the result, the assignment variable Z₍ₙ₎ indicating the presence or absence of treatment, and z₍ₙ₎ (the prediction value 207 for the assignment variable Z₍ₙ₎ indicating the presence or absence of treatment).

That is, since the treatment effect 153 of a patient in the treatment group is highly accurate, when treatment same as treatment to the patient in the treatment group is performed on a patient in the non-treatment group having a value equal to the z₍ₙ₎ (the prediction value 207 for the assignment variable Z₍ₙ₎ indicating the presence or absence of treatment) of the patient in the treatment group, it can be estimated that a treatment effect same as the treatment effect 153 of the patient in the treatment group can be obtained.

### Second Embodiment

Next, the second embodiment will be described. The second embodiment shows another process example of the treatment effect calculation (step S305). In the treatment effect calculation (step S305) according to the second embodiment, the treatment effect 153 is calculated by the following Equation (10) by directly searching for patients to be compared between the treatment group and the non-treatment group in one patient population not clustered in the patient stratification (step S303) or in the clustered patient populations C₍ₖ₎. Here, the patient population C₍ₖ₎ will be described as an example.

argmin₍ⱼ₎|\vec{η}₍ᵢ₎−\vec{η}₍ⱼ₎|_{L2} Equation (10)

The causal relation inference device 120 calculates, by the above Equation (10), a patient j who has the minimum distance L2 between a reallocation vector 205 (η₍ᵢ₎) of a patient i belonging to the treatment group and a reallocation vector 205 (η₍ⱼ₎) of the patient j in the non-treatment group. If the distance L2 is equal to or greater than a threshold value, the patient j is not applicable. In the second embodiment, the above threshold is set to be 3 sigma or more of an average distance between reallocation vectors between different pairs. It should be noted that a pair set including a tuple (i, j) of the patient i and the patient j is set as pairs.

By the treatment effect calculation (step S305) using the above Equation (10), it is possible for a physician or an analyst to intuitively understand an analysis procedure of the causal relation inference device 120 with respect to the first embodiment.

In addition, the causal relation inference device 120 may perform the treatment effect calculation (step S305) in the above Equation (10) by using, instead of the reallocation vector 205, any of the pointwise weight vector 206, the feature vector 201, the propensity score 152, and the patient-specific importance vector p₍ₙ₎ calculated by the above Equation (6).

As described above, by performing the treatment effect calculation (step S305) using the pointwise weight vector 206, the treatment effect 153 can be obtained by emphasizing the pointwise weight vector 206, which is a weight for each patient. Accordingly, a user can easily associate the pointwise weight vector 206 with the treatment effect 153 in the patient population C₍ₖ₎.

In addition, by performing the treatment effect calculation (step S305) using the feature vector 201, the treatment effect 153 can be obtained by emphasizing the feature vector 201. Accordingly, a user can easily associate the feature vector 201 with the treatment effect 153 in the patient population C₍ₖ₎.

In addition, by performing the treatment effect calculation (step S305) using the propensity score 152, the treatment effect 153 can be obtained by emphasizing the prediction value 207 for the assignment variable Z₍ₙ₎ indicating the presence or absence of treatment. Accordingly, a user can easily associate the propensity score 152 with the treatment effect 153 in the patient population C₍ₖ₎.

In addition, by performing the treatment effect calculation (step S305) using the importance vector p₍ₙ₎, the treatment effect 153 can be obtained by emphasizing the importance vector p₍ₙ₎. Accordingly, a user can easily associate the importance vector pᵢn) with the treatment effect 153 in the patient population C₍ₖ₎.

In addition, the causal relation inference device 120 calculates, by using the pairs and by the following Equation (11), a consistent average treatment effect MATE (k) for each patient population C₍ₖ₎ (step S305). It should be noted that |pairs| on a right side of the following Equation (11) is the number of elements of the pairs.

$$MATE ( k ) = \frac{1}{\vert pairs \vert} {\sum }_{( i , j ) ∈ pairs} ( {y}_{( i )} - {y}_{( j )} ) \qquad Equation ( 11 )$$

### Experiments

Here, an experimental example will be described. Non-Patent Literature 4 is a document relating to a method for evaluating an effect of a vocational training program. In order to evaluate performance of the causal relation inference device 120 according to the first embodiment, data in Non-Patent Literature 4 was used. To explain relationships between symbols in the first embodiment and the data in Non-Patent Literature 4, the feature vector 201 corresponded to {age, education, Black (1 if black, 0 otherwise), Hispanic (1 if Hispanic, 0 otherwise), married (1 if married, 0 otherwise), nodegree (1 if no degree, 0 otherwise), RE75 (earnings in 1975)}, and the treatment effect y₍ₙ₎ corresponded to RE78 (earnings in 1978).

In addition, the Z₍ₙ₎ indicating the presence or absence of treatment corresponded to a treatment indicator (1 if treated (treatment), 0 if not treated (control)). An exact therapeutic effect with or without treatment is calculated by NSW data in Non-Patent Literature 4. In addition, data input to the causal relation inference device 120 according to the first embodiment corresponds to CPS-3 in Non-Patent Literature 4.

A case where ATE was calculated after propensity score calculation was performed by using a standard method referred to as logistic regression was compared with a case where ATE (AATE in the first embodiment and MATE in the second embodiment) output by the causal relation inference device 120 according to the first embodiment and the second embodiment was calculated.

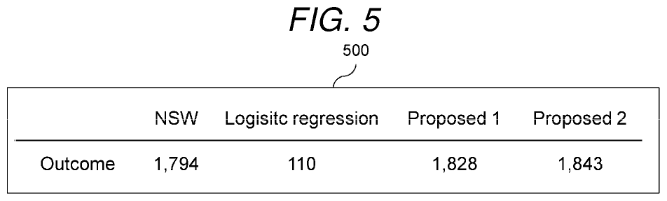

FIG. 5 is an explanatory diagram showing an experimental result. In an experimental result 500, a value of ATE of NSW of $1,794 was set as a true value. A value of ATE of logistic regression was “$110”, a value of ATE (AATE) of Proposed 1 (the causal relation inference device 120 according to the first embodiment) was “$1,828”, and a value of ATE (MATE) of Proposed 2 (the causal relation inference device 120 according to the second embodiment) was “$1,843”.

Therefore, with the causal relation inference device 120 according to the first embodiment and the second embodiment, the treatment effect 153 can be better estimated by a physician or an analyst than a case where the standard method (logistic regression) is used.

FIG. 6 is a diagram illustrating an importance distribution for each factor of a feature using kernel density estimation in the experimental result 500. An importance distribution 601 is a distribution of values of the importance vector p₍ₙ₎ when a factor of the feature of each sample is age. An importance distribution 602 is a distribution of values of the importance vector p₍ₙ₎ when the factor of the feature of each sample is whether the sample is educated or an education level.

An importance distribution 603 is a distribution of values of the importance vector p₍ₙ₎ when the factor of the feature of each sample is whether the sample is black. An importance distribution 604 is a distribution of values of the importance vector p₍ₙ₎ when the factor of the feature of each sample is whether the sample is Hispanic. An importance distribution 605 is a distribution of values of the importance vector p₍ₙ₎ when the factor of the feature of each sample is whether the sample is married.

An importance distribution 606 is a distribution of values of the importance vector p₍ₙ₎ when the factor of the feature of each sample is whether the sample is not graduated from a high school (nodegree). An importance distribution 607 is a distribution of values of the importance vector p₍ₙ₎ when the factor of the feature of each sample is an annual income of 1974 (re74). An importance distribution 608 is a distribution of values of the importance vector p₍ₙ₎ when the factor of the feature of each sample is an annual income of 1975 (re75).

In the importance distributions 601 to 608, a horizontal axis represents a value of each importance vector and a vertical axis represents a kernel density estimator (generally, frequency). In addition, “Treatment” indicates treated samples (treatment group), and “Control” indicates untreated samples (non-treatment group).

For example, according to the importance distribution 606 relating to whether the samples are not graduated from a high school (nodegree), it is understood that when a value of high school non-graduation (nodegree) is “1”, a probability of being treated is increased. Participants in the Control group were not actually treated, but if the participants have an opportunity to be treated, it is highly probable that a sample n having a value of high school non-graduation (nodegree) of “1” is in a group with a high possibility of being treated.

As described above, the causal relation inference device 120 according to the first embodiment and the second embodiment can implement the propensity score analysis with high accuracy and easily explain the analysis process.

In addition, the above-mentioned causal relation inference device 120 according to the first embodiment and the second embodiment can also be configured as described in (1) to (13) below.

(1) In the causal relation inference device 120 including a processor 123 configured to execute a program and a storage device (memory 122) storing the program, the processor 123 executes a first calculation process (step S401) of calculating internal vectors 202 to 204 based on a feature vector 201 of a plurality of samples (for example, patients) and first learning parameters 211 to 213, a second calculation process (step S402) of calculating a reallocation vector 205 (η₍ₙ₎) based on a second learning parameter 214 and the internal vector 202 calculated by the first calculation process (step S401), and a third calculation process (step S403) of calculating a pointwise weight vector 206 (ξ₍ₙ₎) for each of the plurality of samples based on a third learning parameter 215 and the reallocation vector 205 (η₍ₙ₎) calculated by the second calculation process (step S402).

(2) In the causal relation inference device 120 according to the above (1), the processor 123 executes a fourth calculation process of calculating, for each sample, a prediction value (propensity score 152, z₍ₙ₎) for an assignment variable Z₍ₙ₎ to be assigned to the sample, based on the feature vector 201 and the pointwise weight vector 206 (ξ₍ₙ₎) calculated by the third calculation process (step S403).

(3) In the causal relation inference device 120 according to the above (2), the processor 123 executes an update process (step S405) of updating the first learning parameters 211 to 213, the second learning parameter 214, and the third learning parameter 215 by using the assignment variable Z₍ₙ₎ and the prediction value (propensity score 152, z₍ₙ₎) for the assignment variable Z₍ₙ₎ calculated by the fourth calculation process, in the first calculation process, the processor 123 calculates the internal vectors 202 to 204 based on the feature vector 201 and the first learning parameters 211 to 213 updated by the update process, in the second calculation process, the processor 123 calculates the reallocation vector 205 (η₍ₙ₎) based on the internal vectors 202 to 204 and the second learning parameter 214 updated by the update process, and in the third calculation process, the processor 123 calculates the pointwise weight vector 206 (ξ₍ₙ₎) based on the reallocation vector 205 (η₍ₙ₎) and the third learning parameter 215 updated by the update process.

(4) In the causal relation inference device 120 according to the above (2), the processor 123 executes a fifth calculation process (step S305) of calculating an effect (treatment effect 153 {ATE(k), AATE}) obtained by the plurality of samples, based on the prediction value (propensity score 152, z₍ₙ₎) for the assignment variable Z₍ₙ₎ calculated by the fourth calculation process for each of the plurality of samples, the assignment variable Z₍ₙ₎, and a result y₍ₙ₎ obtained by the sample in the assignment variable Z₍ₙ₎.

(5) In the causal relation inference device 120 according to the above (4), the processor 123 executes a clustering process (step S303: patient stratification) of clustering the plurality of samples, and in the fifth calculation process, the processor 123 calculates, for each of a plurality of sample populations C₍₁₎, C₍₂₎, . . . , C₍ₖ₎ obtained by the clustering process, an effect (treatment effect 153 {ATE(k), AATE}) obtained by the sample population C₍ₖ₎, based on the prediction value (propensity score 152, z₍ₙ₎) for the assignment variable Z₍ₙ₎, the assignment variable Z₍ₙ₎, and the result y₍ₙ₎ obtained by the sample in the assignment variable Z₍ₙ₎.

(6) In the causal relation inference device 120 according to the above (5), in the clustering process, the processor 123 clusters the plurality of samples based on the reallocation vector 205 (η₍ₙ₎).

(7) In the causal relation inference device 120 according to the above (2), the processor 123 executes a sixth calculation process (step S305) of generating, based on a distance between a first sample whose value of the assignment variable Z₍ₙ₎ is a first assignment value (treatment) among the plurality of samples and a second sample whose value of the assignment variable Z₍ₙ₎ is a second assignment value (non-treatment) among the plurality of samples, a pair of the first sample and the second sample, and calculating, in the pair, an effect (treatment effect 153 {ATE(k), AATE}) obtained by the plurality of samples, based on a first result y₍ᵢ₎ obtained by the first sample at the first assignment value and a second result y₍ⱼ₎ obtained by the second sample at the second assignment value.

(8) In the causal relation inference device 120 according to the above (7), the processor 123 executes a clustering process (step S303: patient stratification) of clustering the plurality of samples, and in the sixth calculation process, the processor 123 generates, for each of a plurality of sample populations C₍₁₎, C₍₂₎, . . . , C₍ₖ₎ obtained by the clustering process, the pair of the first sample and the second sample, and calculates, in the pair, an effect (treatment effect 153 {ATE(k), AATE}) obtained by the sample population C₍ₖ₎, based on the first result and the second result.

(9) In the causal relation inference device 120 according to the above (7), in the sixth calculation process, the processor 123 generates the pair based on a distance between the reallocation vector 205 (η₍ₙ₎) of the first sample and the reallocation vector 205 (η₍ₙ₎) of the second sample.

(10) In the causal relation inference device 120 according to the above (1), the processor 123 executes a seventh calculation process (step S304, Equation (7)) of calculating, for each of the plurality of samples, an importance vector p′₍ₘ₎(k) indicating an importance of the feature vector.

(11) In the causal relation inference device 120 according to the above (10), the processor 123 executes a clustering process (step S303: patient stratification) of clustering the plurality of samples, and in the seventh calculation process, the processor 123 calculates the importance vector p′₍ₘ₎(k) for each of a plurality of sample populations C₍₁₎, C₍₂₎, . . . , C₍ₖ₎ obtained by the clustering process.

(12) In the causal relation inference device 120 according to the above (2), the assignment variable indicates treatment for the sample.

(13) In the causal relation inference device 120 according to the above (2), the effect obtained by the sample is a therapeutic effect.

It should be noted that the invention is not limited to the above-mentioned embodiments, and includes various modifications and the equivalent configurations within the gist of the scope of the appended claims. For example, the above-mentioned embodiments are described in detail for a better understanding of the invention, and the invention is not necessarily limited to those including all the configurations described above. Further, a part of the configurations according to a given embodiment may be replaced by the configurations according to another embodiment. Further, the configurations according to another embodiment may be added to the configurations according to a given embodiment. Furthermore, a part of the configurations according to each embodiment may be added to, deleted from, or replaced by another configuration.

In addition, the above-mentioned configurations, functions, processing units, processing measures and the like may be realized partly or entirely by hardware, for example, by designing an integrated circuit, and may be realized partly or entirely by software by causing a processor to interpret and execute programs that implement those functions.

The information of programs, tables, and files, and the like to implement the functions may be stored in a storage device such as a memory, a hard disk, or a solid state drive (SSD), or a recording medium such as an integrated circuit (IC) card, an SD card, and a digital versatile disc (DVD).

Further, control lines and information lines that are assumed to be necessary for the sake of description are described, but not all the control lines and information lines that are necessary in terms of implementation are described. It can be considered that almost all components are actually interconnected.

What is claimed is:

1. A causal relation inference device comprising:
a processor configured to execute a program; and
a storage device storing the program, wherein
the processor is configured to execute
a first calculation process of calculating an internal vector based on a feature vector of a plurality of samples and a first learning parameter,
a second calculation process of calculating a reallocation vector based on a second learning parameter and the internal vector calculated by the first calculation process, and
a third calculation process of calculating a pointwise weight vector for each of the plurality of samples based on a third learning parameter and the reallocation vector calculated by the second calculation process.

2. The causal relation inference device according to claim 1, wherein
the processor is configured to execute a fourth calculation process of calculating, for each sample, a prediction value for an assignment variable to be assigned to the sample, based on the feature vector and the pointwise weight vector calculated by the third calculation process.

3. The causal relation inference device according to claim 2, wherein
the processor is configured to execute an update process of updating the first learning parameter, the second learning parameter, and the third learning parameter by using the assignment variable and the prediction value for the assignment variable calculated by the fourth calculation process,
in the first calculation process, the processor calculates the internal vector based on the feature vector and the first learning parameter updated by the update process,
in the second calculation process, the processor calculates the reallocation vector based on the internal vector and the second learning parameter updated by the update process, and
in the third calculation process, the processor calculates the pointwise weight vector based on the reallocation vector and the third learning parameter updated by the update process.

4. The causal relation inference device according to claim 2, wherein
the processor is configured to execute a fifth calculation process of calculating an effect obtained by the plurality of samples, based on the prediction value for the assignment variable calculated by the fourth calculation process for each of the plurality of samples, the assignment variable, and a result obtained by the sample in the assignment variable.

5. The causal relation inference device according to claim 4, wherein
the processor is configured to execute a clustering process of clustering the plurality of samples, and
in the fifth calculation process, the processor calculates, for each of a plurality of sample populations obtained by the clustering process, an effect obtained by the sample population, based on the prediction value for the assignment variable, the assignment variable, and the result obtained by the sample in the assignment variable.

6. The causal relation inference device according to claim 5, wherein
in the clustering process, the processor clusters the plurality of samples based on the reallocation vector.

7. The causal relation inference device according to claim 2, wherein
the processor is configured to execute a sixth calculation process of generating, based on a distance between a first sample whose value of the assignment variable is a first assignment value among the plurality of samples and a second sample whose value of the assignment variable is a second assignment value among the plurality of samples, a pair of the first sample and the second sample, and calculating, in the pair, an effect obtained by the plurality of samples, based on a first result obtained by the first sample at the first assignment value and a second result obtained by the second sample at the second assignment value.

8. The causal relation inference device according to claim 7, wherein
the processor is configured to execute a clustering process of clustering the plurality of samples, and
in the sixth calculation process, the processor generates, for each of a plurality of sample populations obtained by the clustering process, the pair of the first sample and the second sample, and calculates, in the pair, an effect obtained by the sample population, based on the first result and the second result.

9. The causal relation inference device according to claim 7, wherein
in the sixth calculation process, the processor generates the pair based on a distance between the reallocation vector of the first sample and the reallocation vector of the second sample.

10. The causal relation inference device according to claim 1, wherein
the processor is configured to execute a seventh calculation process of calculating, for each of the plurality of samples, an importance vector indicating an importance of the feature vector.

11. The causal relation inference device according to claim 10, wherein
the processor is configured to execute a clustering process of clustering the plurality of samples, and
in the seventh calculation process, the processor calculates the importance vector for each of a plurality of sample populations obtained by the clustering process.

12. The causal relation inference device according to claim 2, wherein
the assignment variable indicates treatment for the sample.

13. The causal relation inference device according to claim 4, wherein
the effect obtained by the sample is a therapeutic effect.

14. A causal relation inference method using a causal relation inference device, the causal relation inference device including a processor configured to execute a program and a storage device storing the program, the causal relation inference method comprising:
causing the processor to execute
a first calculation process of calculating an internal vector based on a feature vector of a plurality of samples and a first learning parameter,
a second calculation process of calculating a reallocation vector based on a second learning parameter and the internal vector calculated by the first calculation process, and
a third calculation process of calculating a pointwise weight vector for each of the plurality of samples based on a third learning parameter and the reallocation vector calculated by the second calculation process.

15. A non-transitory processor-readable recording medium having recorded thereon a causal relation inference program to be executed by a processor, the causal relation inference program causing the processor to execute:
a first calculation process of calculating an internal vector based on feature vectors of a plurality of samples and a first learning parameter,
a second calculation process of calculating a reallocation vector based on a second learning parameter and the internal vector calculated by the first calculation process, and
a third calculation process of calculating a pointwise weight vector for each of the plurality of samples based on a third learning parameter and the reallocation vector calculated by the second calculation process.
